(19) United States
(12) Patent Application Publication
(10) Pub. No.: US 2025/0032044 A1
(43) Pub. Date: Jan. 30, 2025

(54) EEG-GUIDED SPATIAL NEGLECT DETECTION SYSTEM AND DETECTION METHOD EMPLOYING SAME

(71) Applicants: UNIVERSITY OF PITTSBURGH-OF THE COMMONWEALTH SYSTEM OF HIGHER EDUCATION, PITTSBURGH, PA (US); NORTHEASTERN UNIVERSITY, BOSTON, MA (US); THE UNITED STATES GOVERNMENT AS REPRESENTED BY THE DEPT. OF VETERANS AFFAIRS, WASHINGTON, DC (US)

(72) Inventors: Murat Akcakaya, Pittsburgh, PA (US); Deniz Kocanaogullari, Pittsburgh, PA (US); Emily Grattan, Wexford, PA (US); George F. Wittenberg, Pittsburgh, PA (US); Jennifer Mak, Pittsburgh, PA (US); Sarah Ostadabbas, Watertown, MA (US); Xiaofei Huang, Lynnfield, MA (US)

(73) Assignees: UNIVERSITY OF PITTSBURGH-OF THE COMMONWEALTH SYSTEM OF HIGHER EDUCATION, PITTSBURGH, PA (US); NORTHEASTERN UNIVERSITY, BOSTON, MA (US); THE UNITED STATES GOVERNMENT AS REPRESENTED BY THE DEPT. OF VETERANS AFFAIRS, WASHINGTON, DC (US)

(21) Appl. No.: 18/714,863

(22) PCT Filed: Mar. 30, 2023
(86) PCT No.: PCT/US2023/016885

§ 371 Date: May 30, 2024

Related U.S. Application Data
(60) Provisional application No. 63/325,378, filed on Mar. 30, 2022.

(51) Int. Cl. A61B 5/00; A61B 5/378

(52) U.S. Cl. CPC A61B 5/4842; A61B 5/378; A61B 5/742

(57) ABSTRACT

A method of determining an extent of visual spatial neglect of a patient includes providing a software-based test to the patient via a presentation apparatus positioned on the head of the patient and having a display device positioned close and in front of the eyes of the patient. EEG information is collected from the patient during the test via an EEG apparatus positioned on the head of the patient. Portions of the EEG information collected during the test are used to determine the extent of the visual spatial neglect of the patient. An indication of the extent of the visual spatial neglect of the patient is provided.

### CROSS-REFERENCE TO RELATED APPLICATIONS

This application claims priority under 35 U.S.C. § 119(e) from U.S. provisional patent application No. 63/325,378, entitled “AR-BASED EEG-GUIDED SPATIAL NEGLECT DETECTION SYSTEM AND DETECTION METHOD EMPLOYING SAME” and filed on Mar. 30, 2022, the contents of which are incorporated herein by reference.

### NOTICE OF GOVERNMENT FUNDING

This invention was made with government support under grant numbers 1915083 and 1915065 awarded by the National Science Foundation (NSF) and with support from the U.S. Department of Veterans Affairs. The government has certain rights in the invention.

### FIELD OF THE INVENTION

The disclosed concept relates to methods and systems for determining an extent of visual spatial neglect (SN) of a patient, and, more particularly, to systems and methods that utilize electroencephalography (EEG) and augmented reality (AR) to accomplish such tasks.

### BACKGROUND OF THE INVENTION

Visual spatial neglect (SN) is a syndrome characterized by inattention to contralesional stimuli after stroke. Stroke patients with SN usually display inattention to one side of themselves or the environment, e.g., neglecting to shave one side of the face or dress one side of the body. SN is heavily associated with intrahemispheric disconnections of the white matter attention networks, specifically in the frontal-parietal superior longitudinal fasciculus, and some association with interhemispheric disconnections as well. Some research has also correlated lesions in the ventral frontal lobe, right inferior parietal lobe or superior temporal lobe to the manifestation of SN. Left-sided SN following damage to the right hemisphere (with neglect in 26.2% of stroke cases) is most common and more severe as compared to right-sided SN (in 2.4% of stroke cases). This is most likely due to the lateralization of bilateral attention processing domains to the right hemisphere of the brain. A diagnosis of SN is associated with extended hospitalization, an increased risk of falling, and overall poor functional recovery. Current assessments of SN are insufficient to fully identify and measure the syndrome. Early and accurate neglect detection is crucial for informing rehabilitation strategies to promote functional recoveries.

Existing clinical assessments of SN have several shortcomings. The current gold standard for SN assessment is the Behavioral Inattention Test (BIT). The Conventional BIT (BIT-C) test consists of six (6) pen and paper subtests (line crossing, star cancellation, letter cancellation, figure and shape copying, line bisection, and representational drawing). While it is a simple and inexpensive assessment for SN, the test is limited in its ability to account for compensatory head or body movements that patients may have developed post-stroke to adapt to their condition, such as turning the body or tilting the head to see their screens. Some subtests, like the representational drawing, may also be subjectively scored. While quantitative scores are given for each subtest, the ultimate outcome is pass/fail, rather than a grading providing an indication of the severity of neglect. These tests also do not assess patients in a realistic and dynamic environment.

Many developments in improving the efficacy of classic pen-and-paper tests have investigated computerized methods of assessing SN. Computer-based methods have been shown to be more sensitive in detecting the presence of SN than the BIT-C. Reaction time has been shown to be a reliable assessor of SN in these methods and slower reaction times have been correlated with impairments to the frontal-parietal attentional networks in SN patients. The Starry Night Test, a commonly employed computer-based SN test was successful in detecting SN in putative recovered patients, as previously determined in the BIT-C, using reaction times to visual targets among distractors (e.g., see L. Y. Deouell, Y. Sacher, and N. Soroker, “Assessment of spatial attention after brain damage with a dynamic reaction time test,” J. Int. Neuropsycholog. Soc., vol. 11, no. 6, p. 697, Oct. 2005). The reaction times from a computerized version of the classic Posner cueing test have also been used to screen for even subtle SN (e.g., see J. Rengachary, G. d'Avossa, A. Sapir, G. L. Shulman, and M. Corbetta, “Is the posner reaction time test more accurate than clinical tests in detecting left neglect in acute and chronic stroke?” Arch. Phys. Med. Rehabil., vol. 90, no. 12, pp. 2081-2088 Dec. 2009). However, these studies still lack a method to counteract compensatory strategies of a patient, do not estimate the field of view (FOV) of the patient, and produce only a binary (i.e., yes/no) detection result for SN.

More recent computerized methods have used virtual reality (VR) strategies to detect SN. The Virtual Reality Lateralized Attention Test employs an obstacle-course-like paradigm with different levels of targets and unrelated background activity/distractors and participants have to name the visual targets. However, these tests still only provide a binary result of neglect/no neglect. VR has also been used to quantify the volume of neglected space to determine the extent of neglect, but such study did not include the use of distractors. In reality, patients are often in dynamic backgrounds where many environmental distractions compete for attention, thus making it more difficult to isolate targets. Some patients with SN have demonstrated a reduced ability to inhibit distractors. Hence, use of distractors provides a more accurate estimation of the extent of SN as a better representation of a dynamic real life environment.

While VR may be advantageous in assessing neglect as compared to traditional methods/arrangements, these tools are difficult to use for rehabilitation as the patient cannot see the real world while practicing their activities of daily living (ADLs). In examining efforts to use VR in stroke rehabilitation, it remains uncertain whether learning in a completely immersive virtual environment necessarily translates to learning in a real environment. Additionally, the immersion has also been commonly reported to elicit motion sickness which could be due to a number of factors including duration of use, user health, and prior experience with VR.

In more general studies on visual attention, functional magnetic resonance imaging (fMRI) has been a common modality used to examine the functional neural correlates of visual attention. One fMRI study found distinct brain activation patterns in healthy participants performing a line bisection task. Lateralization of activation to the right hemisphere was seen in the fMRI blood-oxygen-level-dependent (BOLD) response when a visual stimulus was shown. Variations in the BOLD signal also differentiated between responses to valid and invalid targets during a modified Posner task. While fMRI is a non-invasive imaging technique with high spatial resolution, it has low temporal resolution and general implementation issues (e.g., high cost, lack of portability, unsuitable for use during ADL).

### SUMMARY OF THE INVENTION:

These needs, and others, are met by embodiments of the disclosed concept that, as a first aspect, provide a method of determining an extent of visual spatial neglect of a patient. The method comprises: providing a software-based test to the patient via a presentation apparatus positioned on the head of the patient and having a display device positioned close and in front of the eyes of the patient; collecting EEG information during the test via an EEG apparatus positioned on the head of the patient; determining from portions of the EEG information the extent of the visual spatial neglect of the patient; and providing an indication of the extent of the visual spatial neglect of the patient.

The presentation apparatus may comprise an augmented reality apparatus.

The method may further comprise determining the existence of the visual spatial neglect of the patient from some of the EEG information prior to determining the extent of the visual spatial neglect of the patient.

Providing an indication of the extent of the visual spatial neglect of the patient may comprise providing a mapping of the visual spatial neglect of the patient.

Determining from the EEG information the extent of the visual spatial neglect of the patient may comprise employing portions of the EEG information in a machine learning classifier to provide the mapping of the visual spatial neglect of the patient.

Providing the software-based test to the patient may comprise displaying visual cues in a dynamic background via the augmented reality apparatus, and collecting the EEG information during the test may comprise matching a corresponding portion of the EEG information to each of the displayed visual cues.

Providing the software-based test to the patient may comprise providing a plurality of frames to the patient, each frame comprising a target. Each frame of the plurality of frames may comprise a number of distractors. The number of distractors may comprise a plurality of distractors, wherein the target is positioned in the frame among the plurality of distractors. The target may be a different color and/or shape than each distractor of the number of distractors. Each frame may comprise the target positioned amongst a background that is transparent to the patient.

The test may be an augmented reality-based version of the Starry Night test.

As another aspect of the disclosed concept, a system for identifying an extent of visual spatial neglect in a patient is provided. The system comprises: a presentation apparatus sized and configured to be fitted to the head of the patient and having a display device configured to be positioned close and in front of the eyes of the patient; and an EEG apparatus sized and configured to be positioned on the head of the patient; and a computing device in communication with the presentation apparatus, the EEG apparatus, the computing device having a controller and an output device in communication with the controller, wherein the controller is programmed to: provide a software-based test to the patient via the display device of the presentation apparatus; collect EEG information during the test via the EEG apparatus; determine from portions of the EEG information the extent of the visual spatial neglect of the patient; and provide an indication of the extent of the visual spatial neglect of the patient via the output device.

The presentation apparatus may comprise an augmented reality apparatus.

The indication of the extent of the visual spatial neglect of the patient may comprise a mapping of the visual spatial neglect of the patient over a field of view of the patient.

These and other objects, features, and characteristics of the disclosed concept, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are provided for the purpose of illustration and description only and are not intended as a definition of the limits of the concept.

### BRIEF DESCRIPTION OF THE DRAWINGS:

A full understanding of the disclosed concept can be gained from the following description of the preferred embodiments when read in conjunction with the accompanying drawings in which:

FIG. 1 is a block diagram of a system in accordance with a non-limiting example embodiment of the disclosed concept;

FIG. 2 is a partially schematic diagram of an EEG apparatus in accordance with a non-limiting example embodiment of the disclosed concept shown positioned on the head of a user;

FIG. 3 is a partially schematic perspective view of a presentation apparatus in accordance with a non-limiting example embodiment of the disclosed concept;

FIG. 4 is a block diagram of a computing arrangement in accordance with a non-limiting example embodiment of the disclosed concept;

FIGS. 5A and 5B are examples frames in accordance with example embodiments of the disclosed concept displayed to a user of a presentation apparatus such as shown in FIG. 3 via a display device of the apparatus in accordance with an example method of the disclosed concept;

FIGS. 6 is a flow chart of a method for determining an extent of visual spatial neglect of a patient in accordance with an example embodiment of the disclosed concept that can be carried out using the system of FIG. 1;

FIG. 7 is an example of a mapping output of the extent of visual spatial neglect of a patient in accordance with a non-limiting example embodiment of the disclosed concept;

FIG. 8 is a perspective view of an input device in accordance with a non-limiting example embodiment of the disclosed concept;

FIG. 9 is a flowchart showing general connections between elements of a particular example of a system in accordance with a non-limiting example embodiment of the disclosed concept;

FIG. 10 is an illustration of a participant using the system of FIG. 9 showing examples frames displayed and timing thereof in accordance with a non-limiting example embodiment of the disclosed concept;

FIG. 11 is a graph showing an offline correction method utilized in an example embodiment of the disclosed concept;

FIG. 12 is a scatter plot of participant target detection performance vs BIT-C score with a linear line of best fit and correlation coefficient R in accordance with an example embodiment of the disclosed concept;

FIG. 13 is a comparison of topographic head plots of power ratios averaged across all trials of all participants within each group in accordance with an example embodiment of the disclosed concept;

FIG. 14 is an illustration of results of Wilcoxon rank sum tests comparing median power ratios between WSN and SN in accordance with an example embodiment of the disclosed concept, wherein blank spaces indicate locations that were significantly different between groups and hatched spaces indicate locations that were not significantly different between groups; and

FIG. 15 is a comparison of estimated FOV plots from two participants in an example embodiment in accordance with the disclosed concept exemplifying moderate prediction accuracy (a) and high prediction accuracy (b).

### DETAILED DESCRIPTION OF THE INVENTION:

It will be appreciated that the specific elements illustrated in the figures herein and described in the following specification are simply exemplary embodiments of the disclosed concept, which are provided as non-limiting examples solely for the purpose of illustration. Therefore, specific dimensions, orientations, assembly, number of components used, embodiment configurations and other physical characteristics related to the embodiments disclosed herein are not to be considered limiting on the scope of the disclosed concept.

As used herein, the singular form of “a”, “an”, and “the” include plural references unless the context clearly dictates otherwise.

As used herein, the term “number” shall mean one or an integer greater than one (i.e., a plurality).

As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are coupled directly in contact with each other. As used herein, “fixedly coupled” or “fixed” means that two or more components are coupled so as to move as one while maintaining a constant orientation relative to each other.

As used herein, the terms “component” and “system” are intended to refer to a computer related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers. While certain ways of displaying information to users are shown and described with respect to certain figures or graphs as screenshots, those skilled in the relevant art will recognize that various other alternatives can be employed.

As used herein, the term “controller” shall mean a programmable analog and/or digital device (including an associated memory part or portion) that can store, retrieve, execute and process data (e.g., software routines and/or information used by such routines), including, without limitation, a field programmable gate array (FPGA), a complex programmable logic device (CPLD), a programmable system on a chip (PSOC), an application specific integrated circuit (ASIC), a microprocessor, a microcontroller, a programmable logic controller, or any other suitable processing device or apparatus. The memory portion can be any one or more of a variety of types of internal and/or external storage media such as, without limitation, RAM, ROM, EPROM(s), EEPROM(s), FLASH, and the like that provide a storage register, i.e., a non-transitory machine readable medium, for data and program code storage such as in the fashion of an internal storage area of a computer, and can be volatile memory or nonvolatile memory.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As previously discussed, early and accurate detection of SN is crucial for informing rehabilitation strategies to promote functional recoveries. Embodiments of the disclosed concept integrate brain imaging through electroencephalography (EEG) and AR technology in order to identify the presence and extent of SN in stroke patients more reliably and accurately than present approaches.

EEG, like fMRI (previously discussed in the Background) is also a non-invasive brain imaging method, but has very high temporal resolution and is relatively inexpensive to use. Time-domain analysis is useful in terms of analyzing EEG data and deep learning methodologies have been used for EEG classification. Certain EEG features have been shown to be associated with SN: (i) on average there is an increase in N100 and P200 responses in the EEG of perceived targets compared to neglected targets in stroke patients, (ii) the N100a EEG component, which is expected around 130-160 ms after a stimulus, does not exist in the EEG of neglect patients in response to contralesional stimuli and, (iii) subcomponents of the P300 event involved in novelty stimuli detection, P3a and P3b, were reduced in amplitude towards contralesional targets in patients with SN compared to without SN. Even very small visual stimuli are able to elicit measurable event-related potentials in EEG that are able to control a brain-computer interface (BCI). Bandpower analysis is a useful method for analyzing EEG data in the spectral domain that calculates the average contribution of five frequency bands, including delta (0-4 Hz), theta (4-8 Hz), alpha (8-13 Hz), beta (13-30 Hz), and gamma (30-45 Hz) bands to the power of the overall signal. Directing attention to external visual stimuli has been correlated with a decrease in alpha power, particularly in the parieto-occipital areas. A study of non-stroke participants found an increase in alpha power in the parieto-occipital space contralateral to unattended visual stimuli. After stroke, however, alpha power may fail to decrease when the eyes are open. There have been few studies using bandpower analysis in stroke patients with SN. One previous study found that alpha power increased in the stroke hemisphere in patients with SN in the baseline period and during cue-orienting periods. The same study showed a similar increase in stroke patients without SN but less asymmetry between hemispheres. No studies have used bandpower measures to identify SN or differentiated spectral features corresponding to fast and slow reactions to visual stimuli in stroke and stroke with SN. Portability, cost-effectiveness, and high temporal resolution make EEG particularly suitable for use in embodiments of the disclosed concept.

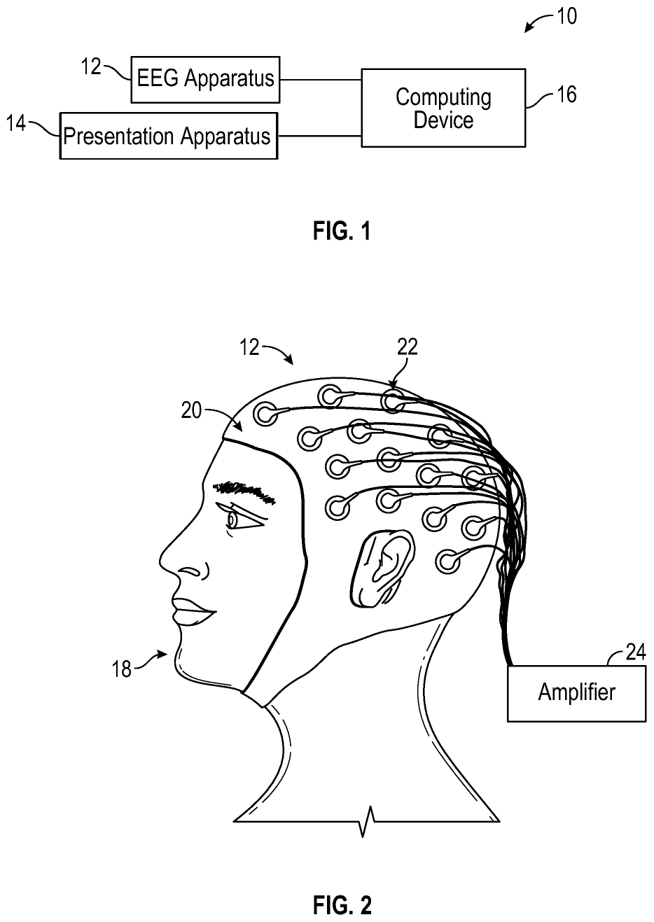

Referring now to FIG. 1, a block diagram of a system 10 in accordance with an example embodiment of the disclosed concept is shown. As briefly discussed above, and described in greater detail below, system 10 is structured to identify, and quantify, SN in stroke patients. System 10 includes an EEG apparatus 12, a presentation apparatus 14, and a computing device 16 in communication (e.g., via suitable wired or wireless arrangement(s) with each of EEG apparatus 12 and presentation apparatus 14.

EEG apparatus 12 is structured and configured to be positioned on the head of a user generally about the scalp of the user and to record EEG signals, also referred to herein as “EEG information”, from the user. A partially schematic diagram of an EEG apparatus 12 in accordance with a non-limiting example embodiment of the disclosed concept shown positioned on the head of an exemplary user 18 is shown in FIG. 2. In such example embodiment, the EEG apparatus 12 includes an EEG cap 20 having a plurality of electrodes 22 (only one is labeled) coupled thereto for collecting EEG information from the exemplary user 18 in a manner such as commonly known. It will be appreciated that this embodiment is meant to be exemplary only, and that other forms of an EEG apparatus 12 may also be used without varying from the scope of the disclosed concept. It is also to be appreciated that one or more of the quantity and/or positioning of electrodes 22 utilized may be varied without varying from the scope of the disclosed concept. As shown in FIG. 2, EEG apparatus 12 may include an amplifier 24 for amplifying signals from the electrodes 22 provided to computing device 16.

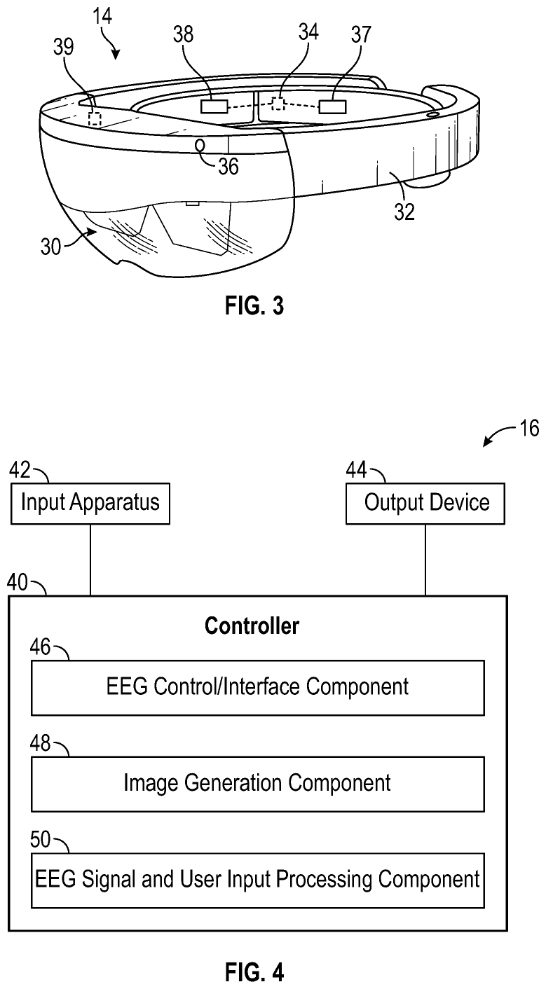

Similar to EEG apparatus 12, presentation apparatus 14 is also structured and configured to be positioned on the head of the user except, as presentation apparatus 14 is utilized (and thus appropriately structured and configured) to display video images consisting of frames to the user as described herein, presentation apparatus 14 is structured and configured to be positioned about the eyes of the user as opposed to the scalp of the user. FIG. 3 is a schematic diagram of presentation apparatus 14 in accordance with a non-limiting example embodiment of the disclosed concept. As seen in FIG. 3, presentation apparatus 14 in this embodiment comprises a head mounted display (HMD) device that includes a display device 30 and a mount 32 that wraps around the head of a user to position the display device 30 generally in close proximity to, and in front of, the user's eyes when providing a virtual reality or augmented (mixed) reality experience to the user. Any suitable display technology and configuration may be used to display visual content via the display device 30. For a virtual reality experience, the display device 30 may be a non-see-through Light-Emitting Diode (LED) display, a Liquid Crystal Display (LCD), or any other suitable type of opaque display. In some cases, an outwardly facing camera 36 may be provided that captures images of the surrounding environment, and these captured images may be displayed on the display device 30 along with computer generated images that augment the captured images of the real environment. For an augmented reality experience, the display device 30 may be at least partially transparent so that the user of the HMD device may view physical, real-world object(s) in the physical environment through one or more partially transparent pixels displaying virtual object representations. For example, display device 30 may include image-producing elements such as, for example, without limitation, a see-through Organic Light-Emitting Diode (OLED) display. By utilizing such HMDs, embodiments of the disclosed concept improve upon past computer-based methods by accounting for compensatory strategies such as previously discussed in the Background. Such compensatory strategies/movements could misrepresent the number of potentially neglected targets and give a false sense of high performance. When fitted correctly, an HMD will always center its screen in the participant's field of view (FOV), thus making embodiments of the disclosed concept more robust to compensatory techniques and FOV problems with a fixed screen such as previously discussed.

Continuing to refer to FIG. 3, presentation apparatus 14 includes a controller 34 for controlling operation of the presentation apparatus 14, in particular in response to signals from computing device 16. An outwardly facing camera 36 that captures images of the surrounding environment, a wireless transceiver 37 for communicating with computing device 16, as well as one or more other sensors 38 for determining orientation and/or movements of the user's head and presentation apparatus 14 may be provided as components of presentation apparatus 14 in communication with controller 34 thereof. Additionally, an eye tracking arrangement 39 may be provided as a component of presentation apparatus 14 for tracking the positioning of a user's eyes. For example, without limitation, such functionality can be used for tracking the user's eyes to detect/further address potential compensatory techniques, or may be utilized as a user input means, for receiving input from the user in particular embodiments. Wireless transceiver 37 may be used for wireless communications (e.g., without limitation, Wifi, Bluetooth) between controller 34 (and thus presentation apparatus 14) and other devices such as discussed further below. In a preferred, non-limiting example embodiment of the disclosed concept, presentation apparatus 14 is an augmented reality (AR) apparatus such as that just described or other suitable arrangement.

Computing device 16 may be, for example and without limitation, a PC, a laptop computer, a tablet computer, a smartphone, or any other suitable device structured to perform the functions/functionality described herein. Computing device 16 is structured and configured to control presentation apparatus 14 (and/or controller 34 of presentation apparatus 14) as described below to selectively display certain arrangements of objects/elements in the user's normal FOV via the display device 30. As will be discussed further below, the combination of such displayed objects/elements displayed via the display device 30 at a given point in time will be referred to herein as a “frame”. It is to be appreciated that objects/elements displayed with a given “frame” may be provided independently from each other (i.e., separately displayed), as a single image (i.e., similar to a slide show), or via any other suitable means without varying from the scope of the disclosed concept. In embodiments wherein presentation apparatus 14 is a VR apparatus, and thus completely obstructs a user's FOV, the content of frames provided via the display device 30 are all that the user sees. In contrast, in embodiments wherein presentation apparatus 14 is an AR apparatus, and thus at most only selectively obstructs portions of a user's FOV, the objects/elements presented in the frames by the display device 30 appear overlaid in the user's FOV over/onto whatever is present in the user's environment and would be normally viewed in the absence of the presentation apparatus 14. In other words, in VR embodiments the background of the frames (i.e., the area not occupied by the objects/elements) is only whatever is produced by the display device 30 (or by default blank screen), while in AR embodiments the background of the frames is whatever lies in the user's FOV in the environment surrounding the user where the user's head is directed.

Computing device 16 is also structured and configured to receive from the EEG apparatus 12 certain EEG information generated in response to frames, and more particularly to a target object provided therein (discussed below), provided by the display device 30 of the presentation apparatus 14. Such certain EEG information is specifically time-locked to the display of the particular target object. As used herein the term “time-locked” means the portion of the collected EEG signal (i.e., the EEG information) produced from the point in time in which the particular target object is presented and extending for a predetermined period of time thereafter. Such time-locked portion of the EEG signal/EEG information is viewed as a “brain response” to the target image, the purpose of which is discussed further below.

FIG. 4 is a block diagram of a computing device 16 in accordance with an example embodiment of the disclosed concept. Computing device 16 includes a controller 40, and depending on the particular embodiment may also include an input apparatus 42 (e.g., without limitation, a keyboard, a Bluetooth clicker-discussed below, etc.), and/or an output device 44 (e.g., without limitation, an LCD or other suitable display, a wired or wireless transmission arrangement, flash drive, port, or any other suitable arrangement). In certain embodiments, a user and/or practitioner is able to provide input into controller 40 using input apparatus 42, and controller 40 provides output signals to output device 44 to enable output device 44 to display and/or otherwise provide information from controller 40, and thus from system 10, to applicable persons (e.g., user, technician, caregiver, etc.). The memory portion of controller 40 has stored therein a number of routines that are executable by a processor of the controller 40. One or more of the routines implement (by way of computer/processor executable instructions) at least one embodiment of the method discussed in detail herein for determining the presence and extent of SN in a user.

Controller 40 thus includes an EEG control/interface component 46 for interfacing with EEG apparatus 12 and receiving signals (i.e., EEG information) therefrom (e.g., directly or indirectly via a suitable arrangement), an image generation component 48 for generating the previously mentioned objects/frames that are displayed to the user that are discussed in detail below, and an EEG signal and user input processing component 50 for processing the EEG signals/information received by EEG control/interface component 46.

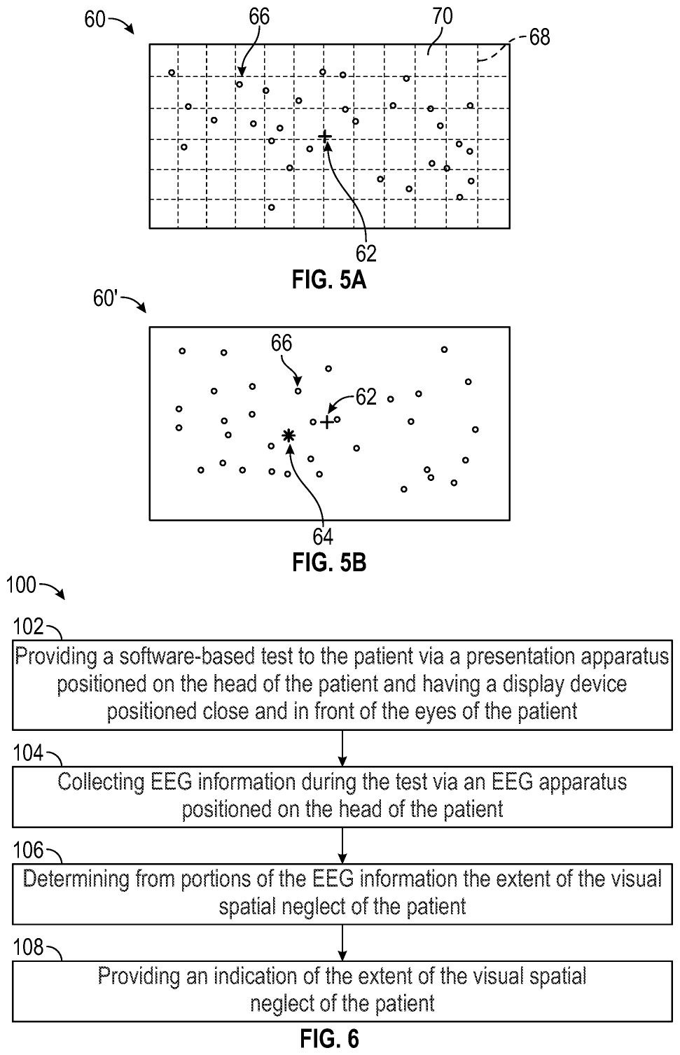

Two non-limiting examples of views/frames 60, 60′ generated by image generation component 48 and presented to the user via the display device 30 of presentation apparatus 14 are shown in FIGS. 5A and 5B. Each frame 60, 60′ is intended to generally encompass the entire FOV of the user and includes a fixation cross 62 which denotes the center of the FOV. In order to not only determine the presence, but more importantly the extent of SN of a user, embodiments of the disclosed concept test the spatial recognition of the user throughout the user's FOV by randomly (from the user's perspective) displaying a target object 64 (FIG. 5B) in different predetermined areas of the FOV, typically, but not necessarily, among a number of distractor objects 66. Each of the distractor objects differ in appearance from the target object 64 in one or more of color, shape, or size. For example, in an example embodiment, a red star/asterisk shaped target object 64 was utilized among green circular shaped distractor objects 66. Such differences between the target object 64 and distractor objects 66 can be readily varied as needed to address needs (e.g., color-blindness) of a particular user being tested.

In the example shown in FIG. 5A, the FOV is shown divided by a reference grid 68 (shown in dashed lines) into an array of individual cells 70, with each cell 70 being one of the aforementioned predetermined areas in which the target object 66 will be displayed at a particular time. It is to be appreciated that reference grid 68 is shown in such example for exemplary purposes only and would not typically be displayed to the user (e.g., see FIG. 5B). In such example, the reference grid 68 divides frame 60 into a 6×12 array of 72 total cells 68. It is to be appreciated that generally any array size may be utilized without varying from the scope of the disclosed concept, however, it is also to be appreciated that while arrays having more cells provides higher resolution, such higher resolution requires subjecting a user to longer testing time (due to the increased amount of cells 70 to be tested), which can lead to less reliable results due to fatigue of the user. Accordingly, in the example shown in FIGS. 5A and 5B the array size was selected due to being a good compromise between resolution and time required for testing

As shown in the example of FIG. 5B, during testing the target object 64 is selectively displayed randomly (from the user's perspective) for short time periods (e.g., without limitation, 0.050 s-0.100 s), one cell at a time in each of the different cells 70, with the previously discussed time-locked EEG information of the user corresponding to each display (i.e., the “brain response” to each display position of the target object 64) recorded for subsequent analysis by the processing component 50 (discussed below). In example embodiments, short time periods (e.g., 1.2 s-2.5 s) are provided between subsequent displays of the target image 64. Such time periods preferably being of random duration. The display locations and durations of the distractor objects 66 are preferably varied during testing. Randomizing the appearance of the target image 64 and distractor images 66 reduces the risk of seizure due to rhythm photic stimulation. The process of displaying the target image 64/distractor images 66 (and recording the corresponding EEG information/brain responses) continues at least until the target image 64 has been displayed at least once in each cell 70. In some example embodiments in accordance with the disclosed concept the process of displaying the target image 64/distractor images 66 is carried out until the target image 64 has been displayed in each cell 70 a predetermined plurality of times (e.g., 2, 3, 4, etc.). Similar to the trade-off previously discussed in regard to selecting array sizing, while increasing the amount of times each cell is tested may increase the accuracy of the end result, the increase in overall test time resulting therefrom can lead to less reliable results due to fatigue of the user being tested.

As mentioned above, the EEG signal and user input processing component 50 of controller 40 processes the EEG signals/information received by EEG control/interface component 46. Such processing can occur generally as such signals/information is received and/or in a number of batches at a later time. In an example embodiment of the disclosed concept, processing component 50 comprises a machining learning-based classifier that has been trained to characterize portions of an EEG signal (i.e., the time locked, “brain response”, portions previously discussed) as being indicative of neglect, not being indicative of neglect, or some degree in between depending on the particular embodiment. It is to be appreciated, however, that processing component 50 may comprise other processing/decision making arrangement(s) without varying from the scope of the disclosed concept.

Having thus described the components of a system 10 in accordance with an example embodiment of the disclosed concept, an example method 100 of determining an extent of visual spatial neglect of a patient (i.e., a user as previously described herein) utilizing system 10 will now be discussed in conjunction with FIG. 6. It is to be appreciated that such method is not limited to the system 10 but may also be carried out with other arrangements having similar functionality/capability without varying from the scope of the disclosed concept. For example, without limitation, method 100 can be carried out using a system/arrangement wherein the functionality of one or more of EEG apparatus 12, presentation apparatus 14, and computing device 16 are provided in a combination arrangement (e.g., similar to a hockey helmet) as opposed to separate elements.

Referring to FIG. 6, method 100 begins at 102 (which presumes the patient/user has already donned EEG apparatus 12 and presentation apparatus 14) by providing a software-based test to the patient via the presentation apparatus 14 positioned on the head of the patient and having the display device 30 positioned close and in front of the eyes of the patient. During the test provided at 102, a target image 64 is provided at locations within the user's FOV as discussed elsewhere herein and EEG information is collected from the patient via EEG apparatus 14 positioned on the head of the patient, such as shown at 104. Next at 106, the extent of the visual spatial neglect of the patient is determined from portions of the EEG information (referred to elsewhere herein as the “brain response[s]”) collected at 104. Finally, an indication of the extent of the visual spatial neglect of the patient is provided, e.g., to a technician, caregiver, user, etc., such as shown at 108.

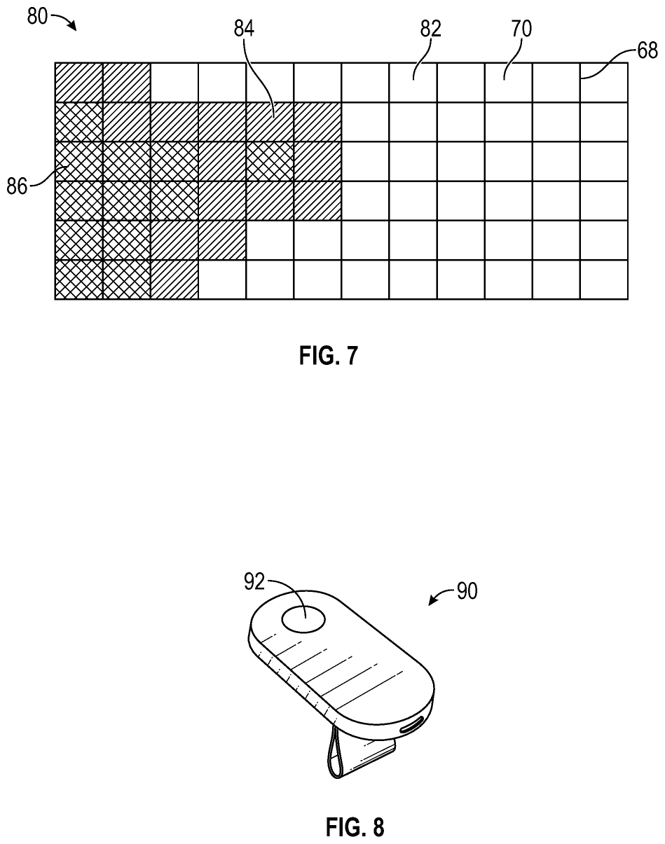

An example of an indication of the extent of the visual spatial neglect of a patient in accordance with a non-limiting example embodiment of the disclosed concept is shown in FIG. 7. In such example, the indication provided is a spatial neglect map 80 showing the extent of spatial neglect in the patient. Like the example frame shown in FIG. 5A from the testing provided at 102 that resulted in the spatial neglect map 80, the map 80 is divided in an array of cells 70 by a grid 68, with each cell containing an indicator of the extent of neglect determined therein. Such indicator(s) may be, for example, without limitation, different colors, shades of greyscale, numerical indicators (e.g., a % indicative of the probability the cell is neglected), hatching, etc. In the example embodiment shown in FIG. 7, each of cells 70 that shows neglect is shown hatched, with those showing the greatest extent being shown as cross hatched, e.g., cell 86; those showing a lesser extent of neglect, e.g., cell 84, being shown as partially hatched; and those not showing neglect being completely unhatched (i.e., no hatching whatsoever). From such map 80, a better targeted rehabilitation plan can be planned/pursued for the patient.

Having thus described a general overview of a system and method in accordance with a general example embodiment of the disclosed concept a more detailed particular example embodiment will now be provided in which testing and proof of the disclosed concept are discussed. The example system discussed in such embodiment is referred to as “AREEN”. It is to be appreciated that such further detailed example is provided for exemplary purposes only and that while details provided in such example may be applicable to other embodiments, such example is not intended to be limiting upon the scope of the disclosed concept.

### I. METHODOLOGY

### A. AR-Based EEG-Guided Neglect Detection (AREEN) System

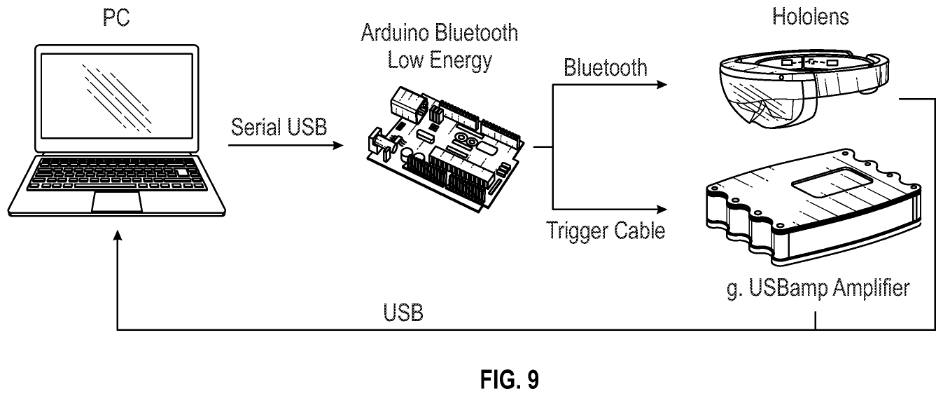

The AREEN system in this example was developed as an integrated multi-modal tool for detection, assessment, and rehabilitation of unilateral SN caused by stroke. It detects and maps visually neglected extra-personal space with high accuracy through continuous EEG-guided SN detection. Unlike previous BCI applications which provide fixed-location visual cues, embodiments of the present system provide a customized application that tracks head position in real-time and projects the holographic visual cues dynamically in the participant's visual space. AREEN records EEG signals as a user views randomly appearing and disappearing targets on an AR headset display. The application itself can be considered as a cascade of multiple applications in different platforms working as a whole (FIG. 9). A modified version of the Starry Night Test (e.g., see L. Y. Deouell, Y. Sacher, and N. Soroker, “Assessment of spatial attention after brain damage with a dynamic reaction time test,” J. Int. Neuropsycholog. Soc., vol. 11, no. 6, p. 697, Oct. 2005) was built specifically for the HoloLens in Unity Plus (Unity, San Francisco, CA, USA). The system interface was built on MATLAB R2015a and the EEG collection module was built on MATLAB R2015a with gTec MATLAB API. The computer and the Microsoft HoloLens application are connected via Bluetooth Low Energy (BLE) connection with an Arduino kit. The test is displayed on the transparent lenses where the targets and distractors are clearly seen without much obstruction to the user's vision.

### B. Time Synchronization

In order to more accurately segment the EEG signal sequence, when the target appears in the HoloLens head mounted display, a personal computer (PC) controls and sends triggers to the HoloLens to present targets and to the EEG amplifier to mark the EEG sequence being received at the same time. However, there is difference in latency between when the wireless HoloLens and wired amplifier receive the triggers as wireless technologies have higher latency than wired arrangements. Therefore, considering this inevitable transmission delay, a time correction algorithm is used to correct the timestamp of the EEG signal marker before data analysis.

The HoloLens head mounted display used in such example embodiment only supported two wireless data transmission modes: WiFi and Bluetooth. To minimize the wireless transmission delay, the performance of both modes of transmission were tested 1000 times in different environments (i.e. a public laboratory room, an open office area, and private home). The latency of a communications network is defined as the time needed to transport information from a sender to a receiver. One of the most commonly used measures of latency is the Round-Trip-Time (RTT), meaning the time for a packet of information to travel from the sender to the receiver and back again. Fitting the HoloLens' data transmission latencies to a beta distribution, we observed that the WiFi network is greatly affected by the environment with a very large range of transmission delay (from about 20 ms to 550 ms), while the Bluetooth transmission method resulted in the lesser delay time (the average of two-way delay is about 75 ms), allowing for more stable communication between the HoloLens and the PC.

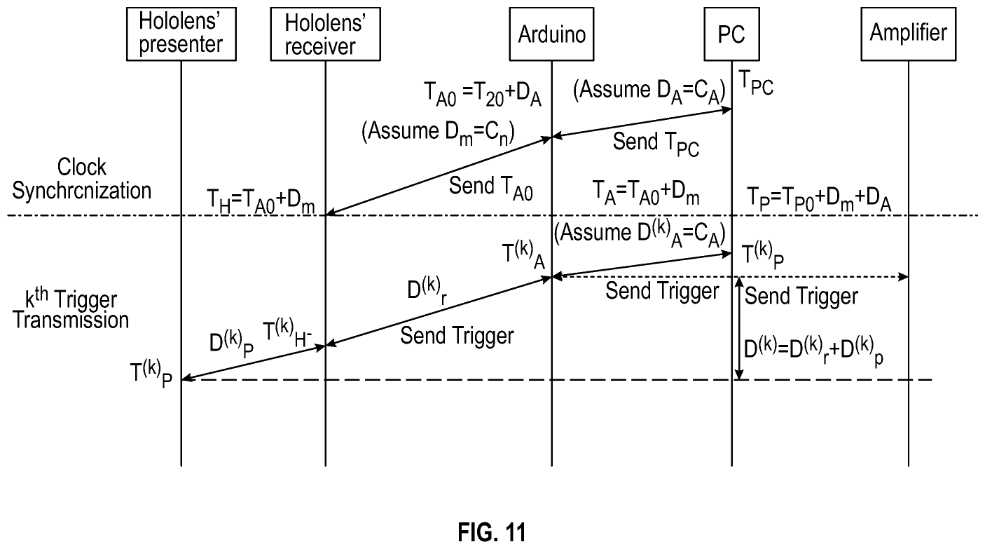

To minimize the impact of asynchronous trigger transmission, an offline correction method was used to correct each trial's EEG marker (FIG. 11), which contains two stages: Clock Synchronization and k^{th} Trial Transmission. In a first stage, the clocks of the PC, Arduino, and HoloLens are synchronized by sending the PC's time to the HoloLens through an Arduino device. The PC's time is defined as universal/reference time. The receipt timestamps of the Arduino and HoloLens based on universal time are noted as T_{A0} and T_{H} respectively. The original time of the HoloLens is defined as T_{O}. The PC first sends its timestamp T_{P0} to the Arduino. Once the Arduino receives the PC's time, the Arduino sends the current timestamp T_{A0} to the HoloLens. As soon as the HoloLens receives the current timestamp T_{A0}, the HoloLens will modify its own time from T_{O} to T_{H}. We note that the transmission delay from the PC to the Arduino is D_{A} and assume that the transmission delay between the Arduino and the HoloLens is constant Dₘ (i.e., the estimated average two-way delay divided by two) at this stage. Hence, using the above nomenclature, the time relationships between the PC and Arduino, Arduino and HoloLens can be written as:

$${T}_{A 0} = {T}_{P 0} + {D}_{A} \qquad ( 1 )$$

$${T}_{H} = {T}_{A 0} + {D}_{m} \qquad ( 2 )$$

After synchronizing the clock (from the central horizontal dotted line in FIG. 8), the respective clocks of the PC, Arduino, and HoloLens share the same time (T_{H}=T_{A}=T_{P}). The offset between the original time of the HoloLens and the PC's time (universal time), noted θ, can be computed:

$$θ = {T}_{H} - {T}_{O} = {T}_{A 0} + {D}_{m} - {T}_{O} \qquad ( 3 )$$

For stage two, target triggers are initiated. The PC first sends the trigger to the Arduino. Once the Arduino receives the trigger, the Arduino records the timestamp and sends the trigger to the HoloLens and amplifier. Based on the universal time, the exact delay for each trial can be easily calculated. For the k^{th} trial, we define that the PC's timestamp of sending is T^{k}ₚ, the Arduino's timestamps of receiving/sending is T^{k}_{A}, the HoloLens receives the trigger at time T^{(k)}_{Hr} and presents the target at time T^{(k)}_{Hp}. Then the Arduino's sending time can be computed as:

$${T}_{A}^{( k )} = {T}_{P}^{k} + {D}_{A}^{k} , \qquad ( 4 )$$

where D^{k}_{A} is the corresponding transmission delay from the PC to the Arduino for the k^{th} trial, and the total delay between the Arduino and the target presentation on the HoloLens is:

$${D}^{( k )} = {D}_{p}^{( k )} + {D}_{r}^{( k )} = {T}_{Hp}^{( k )} - {T}_{A}^{( k )} , \qquad ( 5 )$$

where D^{(k)}ₚ and D^{(k)}ᵣ represent presentation delay and propagation delay respectively.

Since the offset θ is known in (3), the synchronized HoloLens's presentation time T^{(k)}_{Hp} can be written:

$${T}_{Hp}^{( k )} = {T}_{O}^{( k )} + θ = {T}_{O}^{( k )} + {T}_{A 0} + {D}_{m} - {T}_{O} , \qquad ( 6 )$$

where T^{(k)}_{O} is the original HoloLens's time for the k^{th} trial.

According to (4) and (6), we can rewrite (5):

$$\begin{matrix}{D}^{k} = {T}_{O}^{( k )} + {T}_{P 0} + {D}_{A} + {D}_{m} - {T}_{O} - ( {T}_{P}^{( k )} + {D}_{A}^{k} ) \\ = {D}_{m} + ( {T}_{O}^{( k )} - {T}_{O} ) - ( {T}_{P}^{( k )} - {T}_{P 0} ) + ( {D}_{A} - {D}_{A}^{k} ) .\end{matrix} \qquad ( 7 )$$

As the connections between the PC and Arduino and the Arduino and the amplifier are wired, we assume their transmission delays are negligible (DA=D^{K}_{A}≈0). Therefore, based on (7), only the HoloLens's elapsed time from it receiving the synchronization message to when it presents the k^{th} target, the PC's elapsed time from sending synchronization message to sending the k^{th} trigger, and the initial delay Dₘ need to be recorded to compute k^{th} trial delay. After data collection, the trigger markers for each trial can be shifted by transforming the delay D^{k} to sample points:

$$N = ( {D}_{m} + ( {T}_{O}^{( k )} - {T}_{O} ) - ( {T}_{P}^{( k )} - {T}_{P 0} ) ) \times \frac{{F}_{s}}{1000} , \qquad ( 8 )$$

where Fₛ is the sampling rate 256 Hz for our system setting, Dₘ is 37.5 ms, the average two-way delay, which is estimated by testing and statistically analyzing, divided by two.

### C. Participants

226 stroke patients were screened from the community and a University of Pittsburgh Medical Center inpatient rehabilitation facility. Exclusion criteria included severe visual field deficits or cognitive impairments. Participants had at least one stroke, had normal or corrected-to-normal vision, and were over 18 years old. Participants completed the BIT-C. If any BIT-C subtests scores were below each subtest's cutoff or the total score was below 129, the participant was categorized as SN. Five participants with stroke and SN and five participants with stroke without SN (WSN) were recruited. Characteristics of participants studied in this example embodiment are provided below in Table I.

### D. Data Collection

Participants were fitted with the EEG and HoloLens. EEG data was collected through 16 electrodes located at Fp1, Fp2, F3, F4, Fz, Fc1, Fc2, Cz, P1,P2, C1, C2, Cp3,Cp4, O1 and O2 according to the 10-20 system with a sampling frequency of 256 Hz. A ground electrode was placed at Fpz and the reference electrode was placed on the left mastoid process.

Four experimental modes were defined: (1) Signal Check to check the quality of 16 channels' EEG signals in real time by visual inspection; (2) FOV Test, which allows the experimenter to calibrate the FOV in the HoloLens including top, left, central, and right edges; (3) Clicker-Based Assessment for EEG data ground truth generation by identifying the locations in the HoloLens canvas in which stimuli is or is not responded to; and (4) EEG-Based Assessment to assess both the existence and severity of neglect by analyzing the recorded participant's EEG in response to visual stimuli shown on random locations on the HoloLens canvas. Each experimental session began with a signal check to inspect signal quality and an FOV test to ensure proper mounting and positioning of the HoloLens. Participants then performed the Clicker-Based and EEG-Based assessments, taking breaks between tests as necessary. These assessments were completed with the participants facing blank white walls with external distractions minimized.

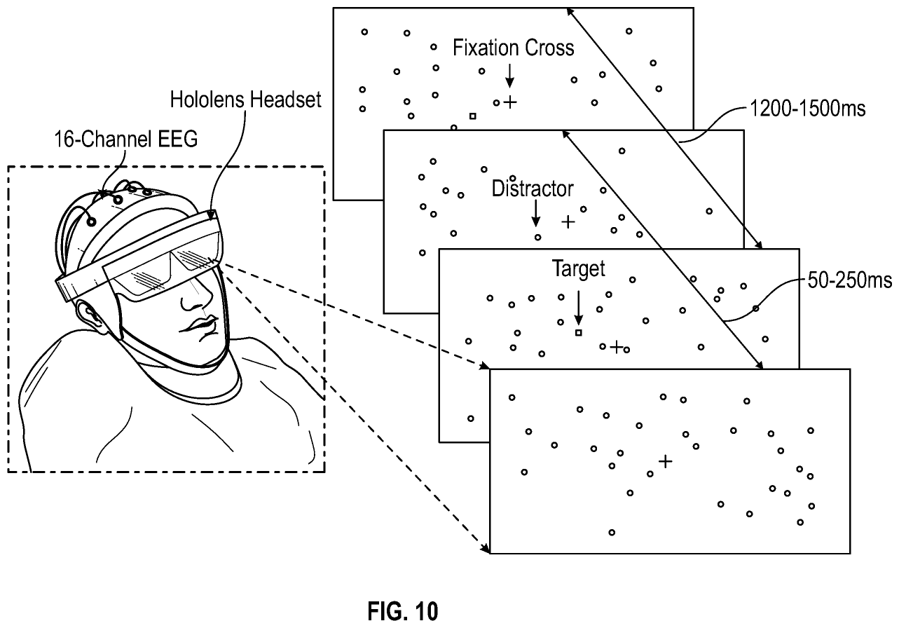

A new paradigm was designed in this new protocol: a modified Starry Night Test for the HoloLens (FIG. 10). The canvas was 0.564 m wide×0.288 m tall, divided into a 6×12 grid (72 total cells) with a fixed depth of 1.14 m. Only one stimulus can occupy one cell at a time. 30-35 distractors, or green stars, were shown at a time for 0.05 s-0.25 s across the grid. Targets, or red stars, were shown one at a time 216 times total (three times in each cell), in a random order for a maximum of 3 s each during the Clicker-Based Assessment and FOV test and 0.066 s each in EEG-Based Assessment. Time between the targets was randomized in the range of 1.2 s-2.5 s. Randomizing the appearance of targets and distractors reduces the risk of seizure due to rhythm photic stimulation. In this study, all participants were able to distinguish between red and green stars. However, it is possible in other embodiments users may experience red-green colorblindness. The colors of the stars are easily reprogrammable in the app developer to address such situation(s).

During the Clicker-Based Assessment, participants had up to 3 seconds to respond to a target using a response button 92 of a remote clicker 90 (FIG. 8) and reaction time was collected. No EEG was collected during this period. During the EEG-Based Assessment, targets appear for a fixed time, as the participant does not give direct input to the system but their EEG is collected. The target number order is recorded for both assessments.

### E. Preprocessing

The EEG data was filtered through an 8^{th} order Butterworth bandpass filter (2-62 Hz) and a 4^{th} order notch filter (58-62 Hz). Data samples were shifted according to the recorded transmission delay times. The data was then segmented into signal and baseline segments, or 500 ms following and 200 ms prior the appearance of a target, respectively. The average baseline amplitude was subtracted from the signal segment in the time-domain for baseline correction. Artifact removal and repairing was completed using the Autoreject algorithm (e.g., see M. Jas, D. A. Engemann, Y. Bekhti, F. Raimondo, and A. Gramfort, “Autoreject: Automated artifact rejection for MEG and EEG data,” NeuroImage, vol. 159, pp. 417-429, Oct. 2017). The total number of trials preserved after Autoreject and therefore used for analyses are detailed in Table I below.

TABLE I
Participant Characteristics

| | | | | | | BIT-C |
|---|---|---|---|---|---|---|
| | | | | | | subtests |
| | | | | Days | | at or |
| | | | Stroke | Since | BIT-C | below |
| ID | Age | Sex | Hemisphere | Stroke | Total | cutoff (/6) |
| SN101 | 81 | F | Right | 701 | 107 | 3 |
| SN102 | 78 | M | Right | 17 | 56 | 6 |
| SN103 | 50 | M | Right | 15 | 112 | 5 |
| SN104 | 61 | F | Left | 9 | 106 | 5 |
| SN105 | 37 | F | Right | 13 | 117 | 5 |
| mean ± SD | 61.4 ± 18.6 | — | — | 151 ± 307 | 100 ± 25 | 5 ± 1 |
| WSN101 | 35 | M | Left | 2404 | 138 | 0 |
| WSN102 | 57 | F | Left | 2466 | 145 | 0 |
| WSN103 | 80 | M | Right | 823 | 142 | 0 |
| WSN104 | 27 | M | Left | 483 | 146 | 0 |
| WSN105 | 73 | M | Right | 15 | 144 | 0 |
| mean ± SD | 54.4 ± 23.1 | — | — | 1238 ± 1130 | 143 ± 3 | 0 ± 0 |

The 6×12 display grid was divided down the middle so that half the cells were on the left and the other half were on the right. For each participant, EEG segments to the targets corresponding to the target location were labeled “ipsilesional” or “contralesional”, based on the lesioned hemisphere for each participant. The EEG data was also labeled with their corresponding band and electrodes. There are a total of 80 possible labels, as there are 16 electrodes and five bands.

For each session, the median reaction times of the three responses per each of the 72 targets were thresholded using a majority-voting procedure. Getting the median increases the number of slow-response targets in a way that is more robust compared to getting the mean in terms of outlier behavior. These times were thresholded using Otsu's method (e.g., see N. Otsu, “A threshold selection method from gray-level histograms,” IEEE Trans. Syst., Man, Cybern., vol. SMC-9, no. 1, pp. 62-66, Jan. 1979). Otsu's method, while mainly used for image binarization, is an algorithm that iteratively searches for a threshold. The selected threshold maximizes the variance (equivalently minimizing the intra-class variance) between two classes: slow-response and fast-response.

For intra-class variance defined as σ²ᵢₙₜᵣₐ(i)=ω₀(i)σ₀²(i)+ω₁(i)σ₁²(i), weights ω_{0,1}are probabilities of the classes separated by a threshold i. σ_{0,1}² are variances of the classes. ω_{0,1}(i) are computed from k-bin histogram:

$${ω}_{0} ( i ) = {\sum }_{x = 0}^{i - 1} p ( x )$$

$${ω}_{1} ( i ) = {\sum }_{x = i}^{k - 1} p ( x )$$

For two classes, as stated above, minimizing intra-class variance is equivalent to maximizing inter-class variance:

$$\begin{matrix}{σ}_{b}^{2} ( i ) = {σ}^{2} - {σ}^{2} \text{?} ( i ) = \text{?} ( i ) {( {μ}_{0} - {μ}_{T} )}^{2} + \text{?} ( i ) {( {μ}_{1} - {μ}_{T} )}^{2} \\ = \text{?} ( i ) \text{?} {( i ) [ {μ}_{0} ( i ) - {μ}_{T} ( i ) ]}^{2}\end{matrix}$$

$$\text{?} \text{indicates text missing or illegible when filed}$$

where ω_{0,1} are class probabilities, μ_{0,1} are class means and μ_{T} is given by:

$${ω}_{0} {μ}_{0} + {ω}_{1} {μ}_{1} = {μ}_{T} \qquad ( 9 )$$

Reaction times above the threshold were labeled as slow responses. Reaction times below the threshold were labeled as fast responses. The fast and slow labels are meant to characterize response segments as high attention and low attention states, respectively.

### F. Neglect Detection

Bandpowers for delta (0-4 Hz), theta (5-8 Hz), alpha (9-13 Hz), beta (14-30 Hz), and gamma (31-45 Hz) bands were calculated for each signal and baseline segment at each electrode channel. The powers of the ipsilesional responses and contralesional responses were combined from all the participants in this analysis. At each electrodeband location, a power ratio was calculated such that each ipsilesional-response power was divided by the average contralesional-response power. The log of these ratios was taken for analyses. This ratio represents neural activation in the ipsilesional response normalized with respect to the average contralesional response. Wilcoxon rank sum tests with Bonferroni-correction were conducted for every electrode within each band to find significant differences in power ratios (n=940 in WSN, n=852 in SN) between the two groups. A logistic regression analysis was performed using the significant electrode-band locations as features to evaluate the ability of these power ratios to separate SN from WSN. Results were validated with 10-fold cross validation with random shuffling.

### G. Response Prediction

A machine-learning based classification algorithm was created to distinguish between slow-response and fast-response targets across SN and WSN participants. Multiple classifiers that are used in EEG analysis were applied: Quadratic and Linear Discriminant Analyses (QDA and LDA) (e.g., see S. Bhattacharyya, A. Khasnobish, S. Chatterjee, A. Konar, and D. N. Tibarewala, “Performance analysis of LDA, QDA and KNN algorithms in left-right limb movement classification from EEG data,” in Proc. Int. Conf. Syst. Med. Biol., Dec. 2010, pp. 126-131), AdaBoost (e.g., see J. Hu, “Automated detection of driver fatigue based on AdaBoost classifier with EEG signals,” Frontiers Comput. Neurosci., vol. 11, p. 72, Aug. 2017.), Random Forest Classifier (RFC) (e.g., see D. R. Edla, K. Mangalorekar, G. Dhavalikar, and S. Dodia, “Classification of EEG data for human mental state analysis using random forest classifier,” Proc. Comput. Sci., vol. 132, pp. 1523-1532 Jan. 2018.), Naïve Bayes (e.g., see J. Machado, A. Balbinot, and A. Schuck, “A study of the naive Bayes classifier for analyzing imaginary movement EEG signals using the periodogram as spectral estimator,” in Proc. ISSNIP Biosignals Biorobotics Conf., Biosignals Robot. Better Safer Living (BRC), Feb. 2013, pp. 1-4.), Multilayer Perceptron (MLP) (e.g., see Y.-P. Lin, C.-H. Wang, T.-L. Wu, S.-K. Jeng, and J.-H. Chen, “Multilayer perceptron for EEG signal classification during listening to emotional music,” in Proc. TENCON IEEE Region 10 Conf., Oct. 2007, pp. 1-3.), and Regularized Discriminant Analysis and Kernel Density Estimation (RDA+KDE) (e.g., see T. Memmott et al., “BciPy: Brain-computer interface software in Python,” 2020, arXiv: 2002.06642.). RDA+KDE was the main classifier worked on and has been demonstrated to work well on event-related potentials (ERP), whereas the other classifiers are used for comparison. However, here a different feature extraction method was used than channel-wise principal component analysis approach, instead common spatial patterns (CSP) was used as the feature extraction algorithm.

Common Spatial Patterns as Discriminative Features: Common spatial patterns (CSP) is an algorithm to calculate spatial filters and it is widely used in BCI systems. It was first proposed to classify imagined hand movements by using multi-channel EEG. The goal is to design a pair of spatial filters such that the filtered signal's variance is maximal for one class while minimal for the other, and vice versa. Let Xᵢ∈R^{N}ᵢ^{×C} be the filtered EEG signals, where i∈1, 2 denotes the class. The algorithm computes a spatial filter ω∈R^{C}

$${max}_{w} \frac{{w}^{T} {X}_{1}^{T} {X}_{1} w}{{w}^{T} {X}_{2}^{T} {X}_{2} w} \qquad ( 10 )$$

As the equation above is invariant with the scale of ω, ∀ω≠0, it can be formulated as:

$$\begin{matrix}{max}_{w} {w}^{T} {X}_{1}^{T} {X}_{1} w & s . t . & {w}^{T} {X}_{2}^{T} {X}_{2} w = 1\end{matrix} \qquad ( 11 )$$

Finally, by applying Lagrange multiplier, this results in the generalized eigenvalue problem:

$${X}_{1}^{T} {X}_{1} w = λ {X}_{2}^{T} {X}_{2} w \qquad ( 12 )$$

To find multiple spatial filters W, solving the simultaneous diagonalization problem:

$${max \text{?}}_{W ϵ ℝ} trace ( Λ )$$

$$\begin{matrix}s . t . & {W}^{T} {X}_{1}^{T} {X}_{1} W = Λ\end{matrix} , {W}^{T} {X}_{2}^{T} {X}_{2} W = 1$$

$$\text{?} \text{indicates text missing or illegible when filed}$$

where K is the number of spatial filters, Λ is a diagonal matrix of shape K×K, and I is the identity matrix. After taking 16 vectors from each trial using CSP, the average power of each vector is extracted and classified. With X being a normal distributed variable, the classification rule can be given as:

$$d \text{?} ( X ) = {min}_{1 \leq k \leq K} {d}_{k} ( X ) \qquad ( 13 )$$

$$\text{?} \text{indicates text missing or illegible when filed}$$

with

$${d}_{k} ( X ) = ( X - {μ}_{k} ) \text{?} {\sum }_{k}^{- 1} ( X - {μ}_{k} ) + \text{?} \vert {\sum }_{k} \vert - 2 l \text{?} {π}_{k} \qquad ( 14 )$$

$$\text{?} \text{indicates text missing or illegible when filed}$$

where μₖ and Σₖ are class mean vector and covariance matrix respectively and πₖ is the unconditional prior probability of observing a class k data. Equation 14 is called the discriminant score for kth class. Using equations 13 and 14 results in using quadratic discriminant analysis (QDA) for classification. If class covariance matrices are assumed identical, i.e. Σₖ=Σ, 1≤k≤K it results in linear discriminant analysis (LDA). When class sample sizes Nₖ are smaller compared to the dimension of the measurement space p, covariance matrix estimates get highly variable. Regularized discriminant analysis (RDA) attempts to overcome this problem by introducing two parameters for estimating the class covariance matrices: λ and γ. λ is used for regularization of individual class covariance matrices towards a pooled estimate. λ=0 results in LDA whereas λ=1 results in QDA. γ shrinks those class covariance matrices towards a multiple of identity matrix. This parameter is used to alleviate the effects of eigenvalue bias: it decreases the larger eigenvalues and increases the smaller ones. The equation wholly becomes:

$${\hat{\sum }}_{k} ( λ , y ) = ( 1 - γ ) {\hat{\sum }}_{k} ( λ ) + \frac{γ}{p} tr [ {\hat{\sum }}_{k} ( λ ) ] I \qquad ( 15 )$$

RDA is the main classifier scope of this embodiment and the calculations are performed on the data from CSP. After getting scores for each datapoint from RDA, kernel density estimation (KDE) is used with a Gaussian kernel to make predictions. The dataset is comprised of 10 participants, where each participant may have multiple experimental sessions. As there are fewer slow responses than fast responses in all participants, the dataset is imbalanced with a 1:7 ratio, which is preserved after Autoreject. All 16 CSP vectors' average powers are used as features in temporal analysis.

### II. RESULTS

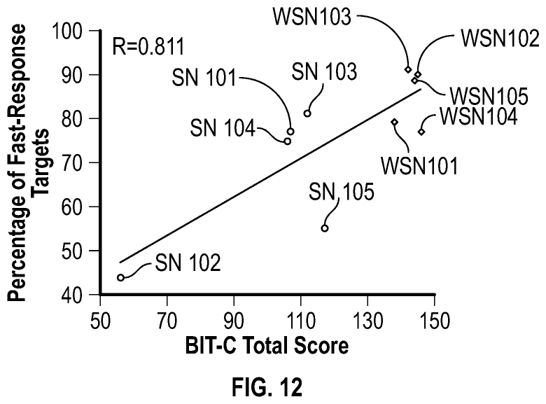

The estimated number of fast and slow responses thresholded from the Clicker-Based Assessment are listed for each participant in Table II below. The percentage of fast responses for each participant has a positive correlation with BIT scores (R=0.811), with a division in performances between SN and WSN (FIG. 12). This demonstrates an expected aspect of performance of the AREEN system.

TABLE II
Target Response Data

| | | | | Median |
|---|---|---|---|---|
| | Fast | Slow | Total | Reaction |
| ID | Responses | Responses | Responses | Time (s) |
| SN101 | 260 | 78 | 338 | 1.42 |
| SN102 | 188 | 240 | 428 | 2.13 |
| SN103 | 164 | 38 | 202 | 1.06 |
| SN104 | 221 | 75 | 296 | 1.48 |
| SN105 | 237 | 195 | 432 | 1.73 |
| mean ± SD | 214 ± 38 | 125 ± 87 | 339 ± 96 | 1.56 ± 0.370 |
| WSN101 | 340 | 90 | 430 | 0.700 |
| WSN102 | 363 | 40 | 403 | 0.892 |
| WSN103 | 366 | 36 | 402 | 0.766 |
| WSN104 | 268 | 80 | 348 | 0.683 |
| WSN105 | 261 | 33 | 294 | 0.801 |
| mean ± SD | 320 ± 27 | 56 ± 27 | 375 ± 54 | 0.768 ± 0.0841 |

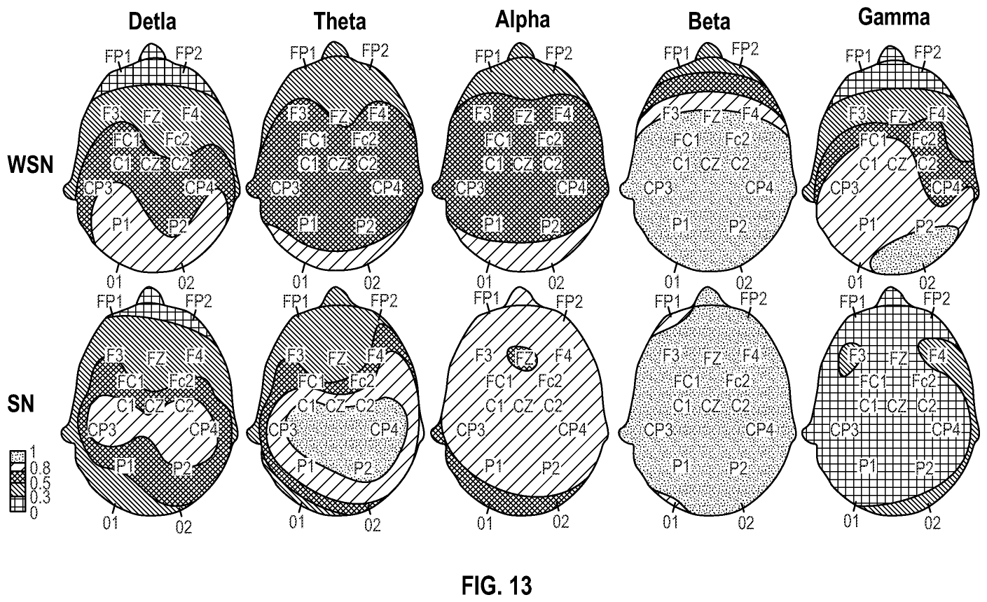

Topographic visualizations of the power ratios reveal similar trends within the two groups' responses to targets across frequency bands, with a few differences in spatial distribution (FIG. 13). Most noticeably, a normalized ipsilesional response within the WSN group is generally most powerful in the highest frequencies (beta and gamma); within the SN group, this is most powerful in theta and beta. In the WSN group, the occipital region is the location of highest power ratio in every band. In the SN group, the points of highest power ratio are concentrated in the central-parietal regions in the lower frequencies (delta and theta) and shifted to the frontal-central regions in the upper frequencies (alpha, beta, and gamma). Both groups see highest power ratios in beta. Between groups, SN generally has higher power ratios in theta and alpha but lower gamma than WSN. In both groups, spatial distributions of power ratio generally vary symmetrically across hemispheres.

### A. Neglect Detection

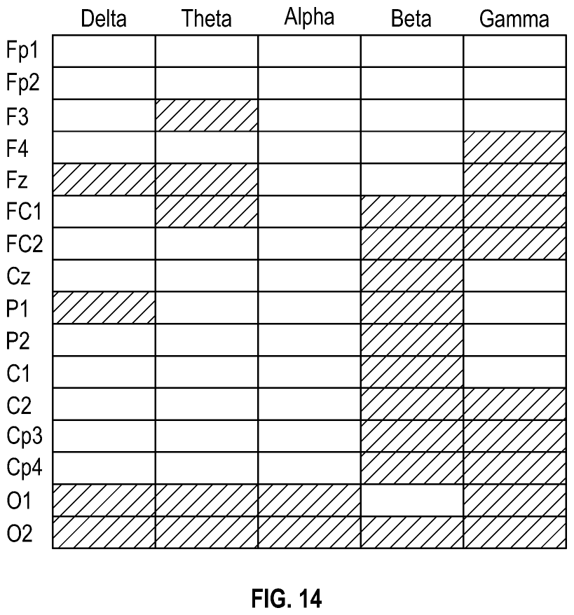

The Wilcoxon rank sum tests using the power ratios found 50 statistically significant (p<0.0006) locations out of the 80 possible locations (FIG. 14). These locations describe nearly the whole brain in delta, theta, and alpha except in occipital electrodes, frontal beta, and mostly left frontal-parietal gamma. These significant areas are symmetric in all bands except gamma. Additional Wilcoxon rank sum tests were performed to compare power ratios in the left and right electrodes within each group and no significant differences were found. The logistic regression analysis using the power ratios at these significant locations yielded an average area under the receiveroperator-characteristic curve (AUC) of 0.853 and 0.832 for the training and testing sets, respectively, demonstrating the high detection probability of an SN participant based on this metric. From the regression, 17 locations were significant, or important to the prediction of neglect, describing frontal-central delta and alpha, frontal-parietal theta, Fp1 beta, and left frontal gamma.

### B. Response Prediction

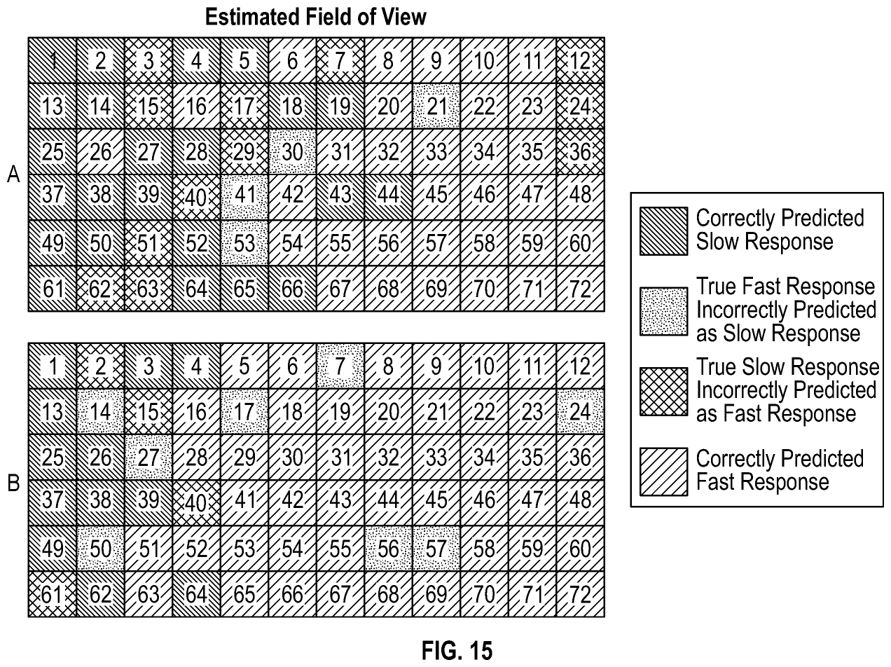

Here, the results for classification between recorded EEG responses corresponding to the slow-response and fast-response targets for the SN and WSN groups are presented to show the performance of the classifier for identification of potentially neglected targets. The results in Table III below are obtained through 10-fold cross validation. The results given in the table are average AUC's over folds. Two examples of the estimated neglected visual fields from participants SN102 (A—FIG. 15) and SN101 (B—FIG. 15), representing the varying degrees in prediction accuracy, are also plotted.

TABLE III
Classification Results

| Method | Average Train AUC | Average Test AUC |
|---|---|---|
| QDA | 0.728 | 0.698 |
| LDA | 0.636 | 0.629 |
| AdaBoost | 0.681 | 0.615 |
| RFC | 0.999 | 0.604 |
| Gaussian Naive-Bayes | 0.717 | 0.716 |
| MLP | 0.816 | 0.613 |
| RDA + KDE | 0.788 | 0.760 |

The results demonstrate that using RDA+KDE shows greater performance compared to other methods whereas RFC and MLP overfits. In this classifier, we only train for RDA, where all λ and γ values are searched and the pair with highest AUC is picked. After getting the ‘best’ λ and γ values, test data is put through RDA with best λ, γ pair and then KDE. The search for λ and γ values are done in a brute force manner, contrary to what is provided with BciPy, where 100 values between 0 and 1 are tried and train AUC are gotten for each fold. BciPy uses constrained optimization by linear approximation algorithm, also known as Powell's method (e.g., see M. J. D. Powell, “An efficient method for finding the minimum of a function of several variables without calculating derivatives,” Comput. J., vol. 7, no. 2, pp. 155-162, 1964.), which does not rely on derivative calculation and −AUC was used as the loss function. We initially chose to search for all the results for our exploratory work where we checked for the impact of λ and γ values.

The temporal analysis takes both slow and fast responses from SN patients and only fast responses from WSN patients. Trials thresholded as slow responses were removed from the WSN group, as slow responses can be considered potentially neglected responses and WSN patients do not have an SN diagnosis. Python was used for temporal analysis and the following libraries: numpy (C. R. Harris et al., “Array programming with NumPy,” Nature, vol. 585, no. 7825, pp. 357-362, 2020), scikit-learn (F. Pedregosa et al., “Scikit-learn: Machine learning in Python,” J. Mach. Learn. Res., vol. 12, pp. 2825-2830, Dec. 2011), MNE (A. Gramfort, “MEG and EEG data analysis with MNE-Python,” Frontiers Neurosci., vol. 7, no. 267, pp. 1-13, 2013), Autoreject, and BciPy (T. Memmott et al., “BciPy: Brain-computer interface software in Python,” 2020, arXiv: 2002.06642).

### III. DISCUSSION

In this example, it has been shown that the AREEN system can feasibly detect SN using the spatiospectral features from EEG responses to visual targets. These features lie within all frequency bands, although there are distinct areas of activation within each band. Important features were distributed across bands and many were located in the frontal area. Significance was also found within the delta band and parietal-occipital areas. Higher power in low frequencies is typically associated with decreased cognitive function and poorer stroke recovery outcomes. This is consistent with the trend seen in power ratios between groups, as the SN group is considered more impaired. Frontal-parietal and parietal-occipital regions are important areas regarding visuospatial attention. Neglect pathology is heterogenous but can generally be correlated with dorsal frontal-parietal network structural or functional dysfunction. The condition is also related to parietal-occipital damage which plays a role in selective attention.

High beta and gamma activity in the left occipital area has been correlated with impaired attention selectivity. From the statistical analyses, we find that the left occipital electrode, O1, had higher power ratios in beta in the SN group compared to the WSN group. However, this is the only electrode representing the entire left occipital cortex, so this outcome should be considered with caution.

Increased task-related alpha activity in the parietal-occipital region is thought to be related to the inhibition of external stimuli, particularly distracting or irrelevant stimuli. Therefore, we could expect to see high alpha power ratio from the WSN group in this region, as they should be better at inhibiting the distractor stars in the Starry Night Task. However, we actually saw greater alpha in the parietal region in the SN group compared to the WSN group and there were no significant differences from the occipital region. This suggests a more general problem at the earliest stages of attention to stimuli, salient or not. Additionally, parietal theta was also higher in the SN group. Although the theta band produced the fewest significant features for neglect detection, the presence of some significant locations in this band expands upon previous findings that did not implicate its importance in identifying post-stroke cognitive deficits. Increased theta and alpha activity have been linked to greater mental fatigue which might account for some of the higher power in these frequencies in the SN group. Participants were encouraged to take breaks between the Clicker-Based and EEG-Based assessments, but at least one participant verbally reported feeling fatigued due to the length of the tests and the requirement to remain still and fixate. Participants in the SN group generally fatigued more and faster than those in the WSN group. This could have further impaired their performance on the task and ability to concentrate.

In this example, power ratio maps within any band tended to be symmetric. Previous research suggests that activation within hemispheres may depend on the location of the stimuli with respect to the lesioned hemisphere. A possible explanation for the departure from the literature is the nature of our metric. The power ratio incorporates responses to both ipsilesional and contralesional targets. Additionally, we recruited both left-hemisphere and right-hemisphere damaged participants in both groups. This study did not control for stroke hemisphere, as the goal of this project was to find general spatiospectral features that would identify SN. Doing so could possibly reveal these hemispheric distribution patterns and improve classification accuracy, but the result may be that we develop a system only applicable for a subset of the neglect population. Future work could investigate using spectral metrics that are robust to inter-individual EEG differences within a group, such as individual alpha frequency, to output more relevant, precise measures for neglect detection or personalized metrics for each patient.

Compared to other related neglect detection systems AREEN more accurately detects the presence and extent of neglect by evaluating its neural signatures rather than behavioral metrics. The system also demonstrates higher accuracy in classifying potentially neglected and observed targets than other solutions and is the first to do so in neglect patients via EEG. The ultimate purpose of the classification algorithms presented in this study is to propose an initial step towards the end goal of rehabilitation of the patient. RDA+KDA, though simple compared to state-of-the-art deep learning models, has demonstrated high AUC values with 10-fold cross validation in our limited dataset. Each fold in cross-validation is stratified; each set is forced to keep the ratio of slow-and fast-response targets. Additionally, given there are five participants with SN and five without SN, one can concur that the model is generalizable. Even though the average results from 10-fold cross validation are good, future translation into rehabilitation requires a highly accurate classifier that can work in the patient's real-world environment.

Although the disclosed concept has been described in detail for the purpose of illustration based on what is currently considered to be the most practical and preferred embodiments, it is to be understood that such detail is solely for that purpose and that the concept is not limited to the disclosed embodiments, but, on the contrary, is intended to cover modifications and equivalent arrangements that are within the spirit and scope of the appended claims. For example, it is to be understood that the disclosed concept contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A method of determining an extent of visual spatial neglect of a patient, the method comprising:
providing a software-based test to the patient via a presentation apparatus positioned on the head of the patient and having a display device positioned close and in front of the eyes of the patient;
collecting EEG information during the test via an EEG apparatus positioned on the head of the patient;
determining from portions of the EEG information the extent of the visual spatial neglect of the patient; and
providing an indication of the extent of the visual spatial neglect of the patient.

2. The method of claim 1, wherein the presentation apparatus comprises an augmented reality apparatus.

3. The method of claim 1, further comprising determining the existence of the visual spatial neglect of the patient from some of the EEG information prior to determining the extent of the visual spatial neglect of the patient.

4. The method of claim 1, wherein providing an indication of the extent of the visual spatial neglect of the patient comprises providing a mapping of the visual spatial neglect of the patient.

5. The method of claim 1, wherein determining from the EEG information the extent of the visual spatial neglect of the patient comprises employing portions of the EEG information in a machine learning classifier to provide the mapping of the visual spatial neglect of the patient.

6. The method of claim 2, wherein:
providing the software-based test to the patient comprises displaying visual cues in a dynamic background via the augmented reality apparatus, and
collecting the EEG information during the test comprises matching a corresponding portion of the EEG information to each of the displayed visual cues.

7. The method of claim 1, wherein providing the software-based test to the patient comprises providing a plurality of frames to the patient, each frame comprising a target.

8. The method of claim 7, wherein each frame of the plurality of frames comprises a number of distractors.

9. The method of claim 8, wherein the number of distractors comprises a plurality of distractors, and wherein the target is positioned in the frame among the plurality of distractors.

10. The method of claim 8, wherein the target is a different color and/or shape than each distractor of the number of distractors.

11. The method of claim 7, wherein each frame comprises the target positioned amongst a background that is transparent to the patient.

12. The method of claim 1, wherein the test is an augmented reality based version of the Starry Night test.

13. A system for identifying an extent of visual spatial neglect in a patient, the system comprising:
a presentation apparatus sized and configured to be fitted to the head of the patient and having a display device configured to be positioned close and in front of the eyes of the patient;
an EEG apparatus sized and configured to be positioned on the head of the patient; and
a computing device in communication with the presentation apparatus, the EEG apparatus, the computing device having a controller and an output device in communication with the controller, wherein the controller is programmed to:
provide a software-based test to the patient via the display device of the presentation apparatus;
collect EEG information during the test via the EEG apparatus;
determine from portions of the EEG information the extent of the visual spatial neglect of the patient; and
provide an indication of the extent of the visual spatial neglect of the patient via the output device.

14. The system of claim 13, wherein the presentation apparatus comprises an augmented reality apparatus.

15. The system of claim 13, wherein the indication of the extent of the visual spatial neglect of the patient comprises a mapping of the visual spatial neglect of the patient over a field of view of the patient.
